(19) United States
(12) Patent Application Publication
(10) Pub. No.: US 2016/0158716 A1
(43) Pub. Date: Jun. 9, 2016

(54) NOVEL RECONSTITUTION ASSEMBLY

(71) Applicant: XSTALBIO LIMITED, Glasgow (GB)

(72) Inventors: JAN VOS, GLASGOW (GB); BARRY DOUGLAS MOORE, GLASGOW (GB)

(73) Assignee: XSTALBIO LIMITED, GLASGOW (GB)

(21) Appl. No.: 14/907,614

(22) PCT Filed: Jul. 28, 2014
(86) PCT No.: PCT/GB2014/052308

§ 371 Date: Jan. 26, 2016

(30) Foreign Application Priority Data
Jul. 26, 2013 (GB) 1313368.1

(51) Int. Cl. B01F 9/00; A61K 38/16; A61K 9/19; B01F 3/12

(52) U.S. Cl. CPC B01F 9/0003; B01F 3/1214; B01F 9/0014; A61K 38/16; A61K 9/19; B01F 2003/1257; B01F 2009/0089

(57) ABSTRACT

The present invention relates to the provision of novel centrifuge activated assembly that can be used for reconstitution of dry formulations on addition of diluent and to pharmaceutical or veterinary products suitable for parenteral administration that have been prepared with or in the devices according to the novel method of the invention.

### FIELD OF THE INVENTION

The present invention relates to the provision of a novel assembly for reconstitution of dry formulations of pharmaceutical or veterinary products. More particularly, the present invention relates to the provision of a novel assembly comprising devices and holders that can be used for sterile reconstitution of dry formulations of pharmaceutical or veterinary products on addition of diluent, and to pharmaceutical or veterinary products suitable for parenteral administration that have been prepared with, or in a novel assembly according to the invention.

### BACKGROUND OF THE INVENTION

Many biopharmaceuticals and some pharmaceutical are formulated in the dry state for reconstitution with diluent prior to parental administration as solutions. However for many biopharmaceutical-containing formulations, reconstitution is problematical, either due to the nature of the active drug molecule itself, for example voluminous dry powders of biomolecules, or from aspects of the formulation, for example the desired concentration level, levels of foam produced or from inconsistencies between reconstituted formulations.

Therapeutic proteins such as monoclonal antibodies are important biopharmaceuticals and there are many therapeutic proteins in development, targeted at a wide range of indications. Typically marketed therapeutic proteins are administered parentally as solutions and treatment may be administered to a subject in hospital via infusion or via injection from a healthcare professional or else be self-administered.

Therapeutic proteins that show poor stability in solution are often stabilized in the dry state. The stabilizing effects may vary from protein to protein but can include reducing mobility, increasing conformational stability and preventing or reducing water catalyzed degradation pathways.

When proteins are stored in the dry state as a dry protein formulation, such as a lyophilized powder or cake they most commonly need to be re-dissolved back into an aqueous diluent before they can be administered to the patient as a solution. The formation of a protein solution on solubilizing a dry protein formulation by addition of a suitable quantity of a diluent, such as water for injection, is generally termed reconstitution. The reconstitution process, beginning with addition of diluent, typically transforms a dry protein formulation from a powder or cake into a solution of the protein. Preferably on completion of the reconstitution process the formed protein solution will be optically clear or else opalescent, but it should not contain any visible particles.

The presence of visible or sub-visible particles may be indicative that degradation processes leading to the formation of protein aggregates have occurred during one or more of the product manufacturing steps, such as filling, freezing, or drying, or during post-manufacture shipping and storage, or else during the reconstitution process itself. It should be noted that great care is taken during the development of marketed therapeutic proteins products to ensure that the risk of producing particles during the manufacture, shipping and storage is minimized. This is because particles present in an administered solution of a therapeutic protein, particularly those that contain denatured protein, are likely to significantly increase the risk of a patient developing an undesirable immune response towards the protein drug. Problems with immunogenicity may include generation of anti-drug antibodies that neutralize or enhance the clearance of the therapeutic protein or else lead to accumulation of the drug. Thus there is a need for a straightforward process for the reconstitution of dry biomolecular formulations, and in particular dry protein formulations, which provides optically clear solutions, without visible particles, and preferably with minimal formation of foam.

The specific process of reconstituting a dry protein formulation is known to carry risks of causing aggregation of the protein which may result in formation of visible or sub-visible particles. Thus, it is well known in the art that shaking or vigorously agitating a protein/diluent mixture can result in a poorly reconstituted product possibly due to shear stresses and/or the production of bubbles which can both cause protein denaturation or unfolding. Thus, commonly the reconstitution instructions provided in the drug product insert of a therapeutic protein, specifically state “do not shake”. Protein solutions that have been shaken or agitated too vigorously typically contain a layer of persistent foam containing air bubbles, released during reconstitution of the dry protein formulation powder or else formed directly by shaking or agitation. Without wishing to be bound by any particular theory it is thought that these bubbles may be stabilized by the presence of denatured protein. Thus there is a need for a process for an improved method for the reconstitution of dry biomolecular formulations, and in particular dry protein formulations, which mitigates the risk of producing visible and sub-visible particle formation.

Whilst the reconstitution instructions provided for dry protein formulations vary in detail from protein to protein the following examples of reconstitution steps-in-common are to be found in the product inserts of the following drugs Synagis® (palivizumab), Herceptin® (trastuzmab), Fuzeon® (enfuvirtide), and Xolair® (omalizumab) which are supplied as dry products in vials: manual reconstitution; swirling (gentle) or rolling (gentle); no shaking; avoidance of foam; clear or opalescent solutions; no particulates.

At present the reconstitution of dry powder formulations of biopharmaceuticals such as biomolecules, antibiotics and therapeutic proteins is typically carried out by hand using aseptic techniques and relies on the skill and experience of the responsible person to ensure the process is reproducibly carried out without degradation of the drug and without contamination by pathogens. Particular care has to be taken to ensure that each of the transfers of diluent or solution between for examples syringes or vials is carried in a way that minimises the possibility of introducing bacteria or viruses into the solution. Furthermore following reconstitution it is generally advised that the solution is administered to the patient within one or two hours or else stored at from 2° C. to 8° C. and used within 24 hours. These precautions are designed to ensure that even if very low levels of contamination have occurred during or after reconstitution the contaminating organism does not have time to multiply to levels that cause a risk before injection.

In order to achieve full reconstitution in a reasonable time, such as less than 30 minutes, it is often stated that it is necessary to “gently swirl the vial”. This is both time-consuming and subjective, and requires the administering person such as the physician to spend time in advance of treating the patient preparing the medicament. Further, if this is done incorrectly with a therapeutic protein it can lead to the formation of “excessive foaming” with the possibility that some small fraction of the protein has been degraded. The person responsible for reconstituting a dry protein formulation has therefore to apply the appropriate aseptic technique and to achieve gentle swirling while also judging whether or not the level of foam produced is excessive or not. Furthermore, if on administration a particular therapeutic protein solution does generate an immune response there is a risk that the patient will henceforth no longer obtain any therapeutic benefit from that drug. In addition, because reconstituted solutions cannot be stored, they cannot be prepared a long time in advance. This means if treatment is delayed the drug may be wasted and it is also not practical for a skilled person such as a pharmacist to prepare a series of reconstituted solutions in advance and pass them onto the patient.

Thus there is a long-felt need for a process for the reconstitution of dry biopharmaceutical formulations, and in particular dry protein formulations, which provides reconstituted formulations suitable for administration to a patient, which can be reconstituted more quickly to reduce wastage, can be reconstituted via a more regulated method to minimize person-to-person differences, and which for proteins in particular, can be reconstituted with reduced risk of foaming.

There is presently a need for suitable reconstitution processes for dry biopharmaceutical formulations which are able to provide a sterile environment for the reconstituted solution. Presently, the addition or transfer of a liquid into a dry powder prior to reconstitution into a solution often involves a multiple-step procedure. For example liquid may need to be transferred from a vial into a syringe and then added to the vessel containing the powder for reconstitution and then following reconstitution the solutions needs be transferred back into a syringe or device for administration to the patient. This is time consuming particularly since all steps need to be carried out using aseptic procedures. There is therefore a need to be able to carry out reconstitution of a dry biopharmaceutical with minimal manual intervention and without requiring use of aseptic procedures.

Thus there is also a need for a process for the reconstitution of dry biopharmaceutical formulations wherein the risk of contamination to the reconstituted solutions is reduced or obviated. Whilst if reconstitution were to be carried out within a dual chamber syringe the initial risk of contamination may be considered to be low at the time reconstitution, however storage of the syringe for later use will significantly increase the risk of contamination. Thus, there is a need for a process for the reconstitution of dry biopharmaceutical formulations in a sterile environment, and in particular for use with products for parenteral administration, such as dual chamber syringes.

It is an object of at least one aspect of the present invention to provide improved methods and, assemblies for reconstitution of dry powders of biopharmaceuticals.

It is an object of at least one aspect of the present invention to provide improved methods and, assemblies for reconstitution of dry powders of biopharmaceuticals with minimal manual intervention.

It is an object of at least one aspect of the present invention to provide improved methods and, assemblies for reconstitution of dry powders of biopharmaceuticals wherein the powders are present in syringes, cartridges or other delivery or storage devices, such as for example vials and the like.

It is an object of at least one aspect of the present invention to provide improved methods and, assemblies for reconstitution of dry powders of biopharmaceuticals to: produce solutions ready for injection; without requiring aseptic procedures; inside sterile packaging.

The Applicants have now developed improved methods for the reconstitution of dry powders of biopharmaceuticals and a novel assembly for said reconstitution.

### SUMMARY OF THE INVENTION

The Applicant has developed a novel method for the reconstitution of dry powders of biopharmaceuticals in which application of a centrifugal force is used to promote, trigger or facilitate the addition or transfer of a liquid diluent into a suitable dry powder and suitable devices for achieving this.

According to a first aspect the present invention provides a novel assembly for the reconstitution of dry products in a centrifuge comprising a device and a suitable holder for the device wherein the holder is suitable for use in the rotor of a centrifuge.

According to an aspect the present invention provides a novel assembly for the reconstitution of dry products in a centrifuge wherein the assemblies comprises a holder for a device wherein the holder is adapted to fit within the centrifuge and wherein the device comprises a chamber suitable for containing a liquid and a further chamber suitable for containing a dry product wherein said chambers are separated by an active partition and wherein the partition is activated upon application of a suitable centrifugal force and the liquid is transferred into the product containing chamber.

According to one aspect the present invention provides a device for use in the assembly for the reconstitution of dry product wherein the device comprises a chamber suitable for containing a liquid and a further chamber suitable for containing a powder wherein said chambers are separated by an active partition and wherein upon activation the two chambers are brought into contact.

According to a further aspect the present invention provides a device for use in the assembly for the reconstitution of dry product wherein the device comprises a chamber suitable for containing a liquid and a further chamber suitable for containing a powder wherein said chambers are separated by an active partition which is activated by centrifugal force to bring the two chambers into contact.

According to a further aspect the present invention provides a device for use in the assembly as defined hereinbefore wherein the chambers in the device are detachable from one another or may be formed as a single structure.

According to a yet further aspect the present invention provides devices as defined herein before wherein the holder is adapted to provide a secure fit for the device when the assembly is placed in the rotor of a centrifuge.

According to a further aspect the present invention provides a method for the reconstitution of a dry product within a reconstitution assembly comprising:
(a) introduction of suitable diluent into a chamber of a device suitable for containing liquid;
(b) introduction of dry product to be reconstituted into a further, separate chamber of the device;
(c) placing the device with separated diluent and dry product containing chambers into a suitable holder and optionally placing said holder within sterile packaging;
(d) placing the device-containing holder in the rotor of a centrifuge and rotating the centrifuge;
wherein the chambers of the device are separated by an active partition; and wherein upon application of a suitable centrifugal force the active partition is activated and diluent is transferred into the product containing chamber.

According to a further aspect the present invention provides a method for the reconstitution of dry product comprising reconstitution of said dry product with a suitable diluent within a device comprising:
(a) introduction of a suitable diluent for the dry product to be reconstituted into a chamber of said device suitable for containing liquid;
(b) introduction of the dry product to be reconstituted into a further chamber of said device suitable for containing dry powder;
(c) placing the device with diluent and dry product containing chambers into a suitable holder in the rotor of a centrifuge and rotating the centrifuge;
wherein said chambers are separated by a partition which brings the two chambers into contact upon application of a suitable centrifugal force; and
wherein during rotation the chambers are located within the centrifuge with the diluent containing chamber located nearer to the centre of the rotor than the dry product containing chamber.

### DESCRIPTION

The assemblies according to the invention in conjunction with the use of centrifugal force promote, facilitate or trigger the transfer or addition of a liquid diluent to a dry product within a device. The devices herein can be used for the reconstitution of a dry product with a diluent with minimal manual intervention and whilst still in a sterile packaging or enclosure. Reconstitution may also be carried out at or below ambient temperature to prevent any loss of integrity of the active components within the reconstituted powder.

For the avoidance of doubt the term dry product as defined herein means any dried product that can be advantageously reconstituted into a diluent by application of a centrifugal force. In particular the present assembly is especially suited to the reconstitution of dry powders. For the avoidance of doubt, all aspects of the present invention herein which refer to dry product particularly include dry powder and/or dry cake. Suitable dry powder formulations and dry cake formulations for use in the device and method according to the present invention include: spray-dried powders or cakes; lyophilised powders or cakes; foams; freeze-spray dried powders; lyophilized protein powders or cakes; precipitated protein powders or cakes; vacuum dried powders or cakes; air-dried powders or cakes; spray dried powders or cakes; and supercritical fluid dried powders or cakes. Advantageously, the present assembly provides for the first time the means to provide a mixture of reconstituted dry products and/or powders in a suitable diluent.

Thus the present invention additionally provides a novel assembly for the reconstitution of one or more dry products in a centrifuge wherein the assembly comprises a holder for a device wherein the holder is adapted to fit within the centrifuge and wherein the device comprises a chamber suitable for containing a liquid and a further chamber suitable for containing a dry product wherein said chambers are separated by an active partition and wherein the partition is activated upon application of a suitable centrifugal force and the liquid is transferred into the product containing chamber.

Suitable dry protein formulations may be prepared according to any of the methods known in the art. Suitable dry powders for use in the devices herein additionally include powders which would benefit from reconstitution with diluent within a sterile packaging.

Preferred dry powders for use in the present devices are dry pharmaceutical formulations, dry biopharmaceutical formulations and dry powders for diagnostic applications. As detailed hereinbefore such dry powders may be prepared by any method including lyophilisation, spray-drying, precipitation or crystallisation or other suitable means.

Examples of dry powder formulations comprising biomolecules for use herein includes both dried formulations of pure biomolecules and dried formulations of mixtures of biomolecules. Formulations of mixtures of biomolecules may include complex mixtures that have been derived from cellular sources such as bacterial lysates that have been made into a dry formulation. Dry biopharmaceutical formulations may contain any suitable therapeutic biomolecule or suitable vaccine. Exemplary dry powder formulations comprising therapeutic biomolecules which may be reconstituted in the devices herein include formulations of peptides, oligonucleotides and/or proteins and mixtures thereof. Exemplary dry powder formulations comprising vaccines which may be reconstituted in the devices herein include formulations of: peptides; proteins; toxoids; nanoparticles; polysaccharides; virus-like particles and live, attenuated or inactivated viruses and bacteria; nucleic acids, lipids, natural biopolymers or synthetic polymers, and mixtures and combinations thereof. Dry powder biomolecule formulations for use in the devices herein may also include acellular formulations, formulations containing live cells or killed cells, attenuated cells or lysed cells or else live or killed viruses. For the avoidance of doubt, where the term biomolecule is used in a general sense herein said term specifically includes proteins in particular.

For the avoidance of doubt the dry powder may be introduced into the powder containing chamber via lyophilisation of a solution within the chamber to produce a lyophilised powder.

According to a preferred aspect the reconstituted solutions prepared in the present devices, in accordance with the present methods are administered via injection, including intradermal, subcutaneous, intramuscular, intravenous, intraosseous, and intraperitoneal.

Examples of dry powder pharmaceutical formulations for use herein include, for example small molecule drugs that require to be administered by injections, such as antibiotics.

For the avoidance of doubt the term diluent as defined herein means liquids suitable for the reconstitution of dry products, and in particular dry powder and/or dry cake formulations. These may be aqueous or non-aqueous diluents. Preferred are diluents which comply with the relevant provisions for inclusion in biopharmaceutical and or pharmaceutical use.

Aqueous diluents are preferred for reconstitution of dry biopharmaceutical formulations and/or dry pharmaceutical formulations. Suitable aqueous diluents for use herein include water for injection (WFI), distilled water, deionised water; sterile water for injection (SWFI); bacteriostatic water for injection (BWFI) i.e. sterile water with a suitable antimicrobial preservative; and buffered aqueous solutions. Aqueous diluents may additionally comprise one or more buffers, surfactants, salts, stabilizers; or mixtures thereof. These additional components may be required to control the tonicity of the reconstituted solution or to stabilise the biomolecule in solution. Buffers, surfactants, salts, and stabilizers suitable for use can be selected from those well-known in the art. The relative amount of aqueous diluent, including where present buffers, surfactants, salts, or stabilizers or mixtures thereon, will be dependent upon the concentration of the target reconstituted solution. Selection of suitable buffers, surfactants, salts and stabilizers for use in any particular aqueous diluent will be dependent upon the particular dry formulation to be reconstituted.

The chambers of the device of the assembly according to the invention may be of any suitable shape or configuration to retain the liquid and dry product(s), or dry powder(s) or dry cake(s) separately and the active partition may be located at any suitable location between said chambers which provides activation upon centrifugation, and the entire assembly, comprising a device and holder as defined herein is of a suitable size for location into a centrifuge rotor. The size and shape of the assembly comprising holders and devices with chambers therein may be changed in order to enable reconstitution of dry product(s), or dry powder(s) or dry cake(s) within centrifuges having different rotor dimensions, or to enable multiple reconstitutions within more than one independent device located separately within either a single holder having means for containing multiple devices at the same time.

Thus, the devices of the present assembly may comprise separate chambers each of which appear similar or identical to a conventional vial, syringe, or cartridge, or mixture thereof, or the devices of the present assemblies may comprise separate chambers within a single container such as a vial, or cartridge, or syringe or a combination thereof wherein the chambers in any such device are separated by an active partition as defined herein. Preferably following centrifugation the device has similar or identical functionality to a syringe or other injection device and can be used to administer the reconstituted solution directly, or to store it safely for later administration.

Advantageously the use of centrifugal force to promote, trigger or facilitate transfer of the diluent in conjunction with use of the novel assembly comprising a holder and dual-chamber/active partition devices as defined herein allows for reduction of the device size versus conventional devices, such as for example dual-chamber syringes which typically require components that need to be manually manipulated to start the reconstitution process. In addition to the economic advantages of reduced device size, the present assembly would additionally allow for multiple reconstitutions at the same time, within a suitable centrifuge with use of a suitable rotor adapted to receive a plurality of devices within holders in accordance with the present novel assembly.

According to a particular aspect the devices within the present assembly comprises chambers may be detachable from each other or may be formed within the same structure. According to a further aspect the holder for the device may be integral with the device or may be detachable, or separate thereto.

The contents of the liquid containing chamber and the dry powder, or dry product containing chamber in the devices of the present assemblies are kept separate from each other by an active partition that keeps the liquid and powder from coming into contact with one another until the application of a sufficient centrifugal force. Once a suitable centrifugal force is applied the active partition allows the liquid to be transferred or added into the dry powder. As discussed hereinbefore the present assembly provides for activation of the device by securement within a suitable holder and placement in the rotor of a centrifuge.

The applicant has found that where such placement is such that when the rotor is rotated at normal centrifuge speed, the powder containing chamber is further from the centre of the rotor than the liquid containing chamber desirable reconstitution is provided for an assembly wherein the active partition is provided by a seal which is ruptured or pierced upon activation. Typically for such assemblies this means the device is placed so that the powder containing chamber is at the bottom of the holder and the liquid containing chamber is above it.

The applicant has found that in assemblies wherein the active partition within the device is for example a stopper valve, desirable reconstitution may be achieved where the device is placed with the liquid containing chamber at the bottom of the holder and the dry product and/or dry powder or dry cake containing chamber above it.

Thus according to a yet further aspect the present invention additionally provides a method for the reconstitution of a dry product within a reconstitution assembly comprising:
(a) introduction of suitable diluent into a chamber of a device suitable for containing liquid;
(b) introduction of dry product to be reconstituted into a further separate chamber of the device;
(c) placing the device with separated diluent and dry product containing chambers into a suitable holder and optionally placing said holder within sterile packaging;
(d) placing the the device-containing holder in the rotor of a centrifuge and rotating the centrifuge wherein the device is placed so that the powder containing chamber is at the bottom of the holder and the liquid containing chamber is above it, or wherein the device is placed with the liquid containing chamber at the bottom of the holder and the dry product and/or dry powder containing chamber above it;

wherein the chambers of the device are separated by an active partition and wherein upon application of a suitable centrifugal force the partition is activated and diluent is transferred into the product containing chamber.

In the method of the invention centrifugation of the present assembly generates a centrifugal force that promotes or triggers a physical change in the active partition between the two chambers of the device and this physical change then allows the transfer of liquid from the liquid containing chamber into the dry product, or dry powder containing chamber to take place. Under normal handling, including during shipping or storage of the present assembly comprising a device, within a holder, the active partition functions as an effective barrier keeping the liquid and dry product and/or dry powder or dry cake separated from each other until reconstitution is desired and the assembly is placed on the rotor of a centrifuge and centrifugal force applied. In addition to activation of the active partition, the rate of transfer of liquid between the chambers may also be actively promoted by the use of centrifugal force. Once the active partition has been activated, by rupture, piercing, opening or other such means, the continued application of centrifugal force to the present assembly generates an increase in the hydrostatic pressure between the two chambers causing the liquid to flow more rapidly from the liquid containing chamber into the dry product, and/or dry powder or dry cake containing chamber. For example, in an assembly as defined herein on transfer of the liquid into the dry powder the centrifugal force applied to the mixture will preferably also promote the reconstitution of the dry powder into the liquid in a shorter time than possible using conventional reconstitution methods, and with minimal or no foaming.

WO 2013/093525 describes the use of centrifugal force to promote the reconstitution of products using conventional vials. The description in WO 2013/093525 of the promotion of reconstitution via use of centrifugal force, are included herein by reference.

Centrifugal forces suitable for use with the assembly and/or methods according to the present invention may typically be obtained with commonly available centrifuges and may be in the range of from about 10 to about 10000 xg. Preferably the active partitions within the devices of the present assembly may be activated by centrifugal forces in the range of from about 50 to about 5000 xg, and especially in the range of from about 400 xg to about 4000 xg. Forces of 400 xg and above are particularly advantageous for the reduction of accidental activation during manufacture, transport and distribution processes, and other processes which the assembly may be subjected to prior to placement into a centrifuge.

Reconstitution of a dry powder containing a biopharmaceutical or pharmaceutical product in dry form as defined herein means the transfer of the dry product from the dried to the liquid state wherein the so-formed liquid appears optically clear to the naked eye and exhibits minimal or no foaming, particularly in the case of biopharmaceutical materials. For the avoidance of doubt, minimal foaming includes solutions which are substantially foam free. The presence of a few bubbles at the solution surface or within the solution is not considered to constitute persistent foam and is included within the definition of minimal or no foam or a substantially foam free solution.

The device for use in the assembly of the invention comprises an active partition or barrier that lies between the at least one chamber containing the liquid diluent (liquid containing chamber) and the at least one chamber containing the dry product and/or dry powder or dry cake (dry product containing chamber). An active partition as defined herein means a barrier that allows the transfer of the liquid or diluent from the liquid containing chamber into the dry product or dry powder or dry cake containing chamber to take place only following application of a sufficient centrifugal force. For the avoidance of doubt, the active partition may comprise a physical barrier adapted to change in permeability or integrity once a centrifugal force has been applied to enable transfer of liquid through channels created in the so-changed barrier, or the active partition may be a physical barrier designed to change position within the device from a closed to an open position upon application of a centrifugal force to enable transfer of liquid. Independent of the specific nature of the physical barrier/active partition within the device of the present assembly, it is the co-operation between the active partition and the application of a suitable centrifugal force which promotes, triggers or facilitates a change to the barrier properties of the active partition to enable liquid or diluent to transfer from the liquid containing chamber into the dry product or dry powder or dry cake containing chamber.

Typically following application of a suitable centrifugal force the liquid is able to pass by, around or through the active partition within the devices for use in the assembly according to the invention.

A unique advantage of using the present assembly comprising devices in conjunction with a suitable holder and centrifugal force to promote, trigger or facilitate the transfer of a diluent into a dry powder is that the transfer process can be carried out under sterile conditions. To-date it has not been possible to do this, and this represents an important step forward in enabling treatment providers, careers and physicians to prepare sterile reconstituted products for direct or parenteral injection without the inherent risks of contamination concomitant with current processes.

As detailed hereinbefore the present assembly provides a device that may be adapted for secure placement within a suitable holder to allow the liquid to powder transfer and subsequent reconstitution of the dry product or dry powder or dry cake within the dry product containing chamber to be carried out within a centrifuge wherein the assembly is sealed within sterile packaging. It is recognised that the operator may need to manipulate the device within the holder and sterile packaging, prior to placement into the centrifuge, in order to alter an aspect of the active partition so that it will be susceptible to the application of the centrifugal force. For example, the active partition may need to be released from a fixed shipping position so that it will move within the centrifuge. However, the Applicants have found that such manipulation may be carried out without compromising the integrity of the sterile packaging. The device may conveniently be supplied in or with a container or holder that fits snuggly into the rotor of a centrifuge such as for example a plastic tube with an external shape similar to a conical centrifuge tube, a so-called Falcon tube having at least one end adapted to fit into the rotor of a centrifuge, the so-called pointed end. The packaging and holder may contain a transparent window so that it is possible to check that transfer of liquid into the powder has not accidentally taken place during shipping.

For the avoidance of doubt, any suitable size of holder may be used in accordance with the present assembly, provided that such holder can be held securely with the rotor of the centrifuge to be used for the reconstitution of dry product in the device held within the assembly. The applicant has found that smaller assemblies have cost and transport advantages, and that so-called Falcon tubes of 15 ml or less are especially useful for use herein. Thus the present invention additionally provides an assembly comprising a device and holder as defined hereinbefore, and in accordance with all additional aspects and variations defined hereinbefore which is adapted to allow the liquid to dry product or dry powder transfer to be carried out within a centrifuge with said device and holder sealed within a sterile package.

Thus according to a yet further aspect the present invention provides an assembly comprising a device and holder as defined hereinbefore wherein the device provides for the sterile reconstitution of dry products comprising a chamber suitable for containing a sterile liquid and a further chamber suitable for containing a dry powder wherein said chambers are separated by a sterile active partition which is activated by centrifugal force to bring the two chambers into contact wherein the device and holder are sealed in sterile packaging.

According to a further aspect the assembly comprising a sterile device and holder may be placed into a centrifuge whilst within unopened sterile packaging and a centrifugal force used to promote, trigger or facilitate the transfer of the liquid into the dry powder under completely sterile conditions. Thus, if the device and contents and holder have been manufactured and packaged under sterile conditions or alternatively sterilised post manufacture using techniques known in the art the use of the present reconstitution process will not compromise this sterility and the reconstituted mixture or solution will remain sterile. This is a significant advantage because sterile reconstituted material may be stored for a much longer period prior to use. Thus instead of needing to be used within 24 hours the reconstituted solution may be kept for much longer prior to administration to a subject.

Depending on the stability of the reconstituted biopharmaceutical or pharmaceutical product in the solution it may be possible to keep it for days, weeks, months or even years prior to administering to a subject. This is particularly envisaged for products which have been reconstituted under sterile conditions as detailed hereinbefore. For example, a protein drug or vaccine in a sealed package could be reconstituted into an aqueous solution in a centrifuge at one site such as a pharmacy or physician's office and supplied to the patient for self-administration at home days, weeks or even months later. Because multiple devices may be reconstituted together in a centrifuge many doses of a biopharmaceutical may be prepared simultaneously. A patient may therefore be efficiently provided with many sterile doses of their medicine, to take at later times, following a single visit to the pharmacist or physician.

The assembly according to the present invention may be used for the reconstitution of dry powders at any suitable temperature, as may be dictated by the stability of the biopharmaceutical or pharmaceutical material, and/or the particular liquid or diluent to be used for such reconstitution. Typically ambient or room temperature use is anticipated, for ease-of-use by physicians or pharmacists. However for some biopharmaceutical or pharmaceutical materials it may well be preferable carry out the reconstitution within the present devices at lower temperature levels to ensure the drug integrity is retained. As such, the present invention additionally provides an assembly for the reconstitution of dry powders as detailed hereinbefore wherein the centrifuge contains a refrigerated rotor. Any suitable centrifuge having a refrigerated rotor or chamber may be used to affect the reconstitution process at below ambient temperature.

In one type of assembly herein the device includes an active partition which is a seal on the liquid chamber which on application of sufficient centrifugal force is ruptured. Alternatively, the liquid chamber may be rigid such as a glass or plastic syringe, cartridge or vial or else may be flexible such as a foil sachet or blister. In either case sufficient centrifugal force may be applied such that the hydrostatic pressure generated by the liquid is sufficient to cause the seal to rupture or break and for the liquid to be transferred into the dry product or dry powder. FIG. 1 shows how such a device may operate.

The hydrostatic pressure in a liquid can determined using the following equation:

p=hpg (1)

wherein p=pressure (N/m², Pa), h=depth at which the pressure is measured (m), p=density of liquid (kg/m³) g=the gravitational constant (9.81 m/s²).

Thus, application of a centrifugal force can be used to significantly increase the hydrostatic pressure of water held in a chamber within the device. With a 1 cm column of water a hydrostatic pressure of about 1 bar may be generated using a centrifugal force of 1000 xg. During shipping and storage the hydrostatic pressure will typically only be 1/1000 to 1/250 of this. Advantageously the active partition may comprise a seal which can be ruptured with a hydrostatic pressure of between 0.1 bar and 10 bar on application of a suitable centrifugal force to the device. Seals which rupture in the hydrostatic pressure range of 0.1 bar to 10 bar are advantageous because they are unlikely to break accidentally but can be activated by pressures accessible with a simple bench-top centrifuge.

The present invention additionally provides an assembly comprising a device as detailed hereinbefore wherein the active partition within the device comprises a seal which can be ruptured with a hydrostatic pressure of between 0.1 bar and 10 bar.

An alternative method of changing the integrity of the seal on the liquid chamber is inclusion of a movable sharp element within the device such as a blade or needle. The centrifugal force may then be used to induce a change in position of either the movable sharp element, or the sealed liquid container within the device or holder so that the needle or blade pierces or cuts the seal of the liquid container and facilitates the transfer of the liquid into the dry powder. If the liquid chamber is a pouch or sachet the movable sharp element may simply pierce or cut the wall of the chamber. Where the liquid containing chamber is a sealed vial and the dry product or dry powder containing chamber is a syringe with a needle centrifugal force can be used to promote piercing of the septum of the vial has shown in Example 1 and as illustrated by FIG. 3.

Preferably the piercing or cutting of the seal (activation of the active partition) is attained by application of a relative centrifugal force in the range 400 xg to 4000 xg. As discussed hereinbefore, centrifugal forces of this intensity can be generated in a standard bench-top centrifuge but will not be experienced by the device under normal storage, handling or shipping conditions so that accidental rupture should not take place. A transparent window on the packaging may be included to enable checking that premature reconstitution has not occurred prior to placing in the assembly in the centrifuge. The rupture of a flexible liquid container such as a sachet containing water for injection may be advantageous because such containers would provide a cost effective method of storing and shipping the liquid diluent.

It is envisaged that the active partition between the liquid containing chamber and the dry product or dry powder containing chamber within the device of the present assembly may also be conveniently achieved by using a novel or existing design of plug or plunger. This may be made from a standard plunger or stopper material such as an elastomer, rubber or thermoplastic or combinations thereof. Such materials are commonly used to form the tips of plungers within hypodermic syringes or cartridge devices or the stoppers of vials. The plug or plunger of the invention is designed to function in a similar way to a valve. On application of a suitable centrifugal force it will allow liquid to pass from the liquid containing chamber into the dry product, or dry powder or dry cake containing Chamber but not vice-versa. The plug or plunger may therefore contain one or more channels passing through it that are designed to open up on application of a suitable hydrostatic pressure on the side of the plunger that is facing towards the liquid containing chamber. The same channels are designed to close up if a similar hydrostatic pressure is applied on the side of the plunger that is initially in contact with the dry product, or dry powder or dry cake containing chamber. This valve property of the active partition means that once liquid has been transferred from the liquid containing chamber to the dry product or dry powder or dry cake containing chamber and reconstitution is complete it can be used as a plunger to expel the reconstituted liquid out of the other end of the product or powder or cake chamber. It is understood that in this invention centrifugal force is used to generate a significantly increased hydrostatic pressure in the liquid containing chamber and this forces open the channels in the plug or plunger and facilitates the transfer of the liquid into the dry product or dry powder containing chamber.

As shown in Example 2, and as illustrated in FIG. 4, with this assembly design for the device and holder surprisingly the powder chamber may be placed so it lies nearer to the centre of the rotor than the liquid chamber on application of a centrifugal force. Thus, the centrifugal force acting on the plunger or stopper valve can be used to generate the required hydrostatic pressure such that channels are opened in the valve and the liquid passes upwards towards the rotor as the valve moves downwards. Preferably the opening of the stopper of plunger valve (activation of the Active partition) is only attained by application of a relative centrifugal force in the range 400 xg to 4000 xg. This will ensure that under storage or shipping the channels will remain closed. Simple experimentation may be carried out to determine an optimal centrifugal force in the preferred range. Preferably the centrifugal force selected will result in transfer of the liquid into the powder chamber in less than 5 minutes and more preferably in less than 2 minutes. Centrifugation of the device within the holder may then be continued until full reconstitution of the dry powder has taken place. The time required will depend upon the type of powder used and the target concentration to be achieved but can be determined by simple experimentation. Many designs of channel may be suitable for the stopper or plunger valve.

It is understood that within the present assembly comprising a device and holder the liquid containing chamber and the dry product or dry powder containing chamber may both be within the same barrel of a syringe or cartridge with the active partition between them formed by a plunger or plug able to function as a valve. Whilst in the centrifuge the plunger should be fixed in place so that the hydrostatic pressure will force the liquid through the valve without moving it. This can be conveniently achieved by designing the holder with internal surface flanges or inserts into which the syringe plunger rod and barrel can be located as shown in FIG. 3. On removal from the centrifuge and holder the plunger may be released so that it can be used in a conventional manner to expel the reconstituted solution from the syringe or cartridge. It is understood that the dry powder, of for example a drug, will be reconstituted with diluent in the chamber of a syringe or cartridge whilst it is being centrifuged. On removal from the centrifuge and from the holder, and upon release of the plunger, the syringe or cartridge may be used to inject the reconstituted drug into a patient.

In some cases the use of an injection device may be required with the above described two chamber syringes or cartridges particularly if the reconstituted drug solution is viscous or for self-injection. Transfer of the syringe or cartridge into the injection device may take place before or after centrifugation. Thus, it is recognised that it may be convenient to place or locate a prefilled, centrifuge-activated, two chamber syringe or cartridge within an injection device prior to centrifugation. The injection device may also be designed to fit into directly into the rotor of the device and thus additionally function as the holder. Placement of the prefilled syringe or cartridge into the injection device may take place during manufacture or post-manufacture but in either case the entire injection device may be placed in a centrifuge and centrifuged in order to reconstitute the dry powder. It recognised that other designs of injection devices that contain a chamber (Liquid Chamber) that contains a liquid and a chamber (Powder Chamber) that contains a dry powder, separated by a partition which is activated by a centrifugal force, may be envisaged, and these are understood to form part of the invention.

Thus the present invention provides means for use of presently approved two-chamber syringed, cartridges or pen devices within the present assembly and for the reconstitution of the dry products therein in accordance with the present method. In another aspect of the invention, the method of altering the partition between the Liquid Chamber and Powder Chamber such that the liquid may be transferred is for the partition to change its position in the device on application of a centrifugal force. Thus the partition may be a plug or plunger held firmly in place during the initial manufacture, shipping and storage. In this position the partition forms a very good seal such that no liquid can be transferred. The plug or plunger may be held in place by frictional forces or may be prevented from moving by a mechanical, electrical or magnetic mechanism which may be released prior to transfer of the device into the centrifuge. When an assembly as defined herein comprising a device in a suitable holder is transferred into the centrifuge sufficient centrifugal force may be applied to move the plug or plunger into a second position in the device (bypass position). In this bypass position there is no longer a seal between the chambers and one or more channels are available for the liquid to pass from the Liquid Chamber into the Powder Chamber. This type of approach based is illustrated in FIG. 5 and is demonstrated in Example 3. Advantageously the transfer of liquid through the channels will be also be facilitated by the hydrostatic pressure provided by the centrifugal force acting on the end stopper or plunger. Preferably the movement of the plunger or stopper into the bypass position (activation of the Active partition) is only attained by application of a relative centrifugal force in the range 400 xg to 4000 xg. This will ensure that under storage or shipping the channels between the chambers will remain closed.

Surprisingly it has been discovered (Example 3) that if the centrifugal force applied is too high the stopper or plunger moves past the bypass position and forms a seal again below it preventing the transfer of the liquid into the dry powder. An optimal centrifugal force should therefore be determined by simple experimentation for the particular type of device and plunger combination. Once determined this optimal centrifugal force may be applied for any similar device in each reconstitution process. Preferably the centrifugal force selected will result in transfer of the liquid into the powder chamber in less than 5 minutes and more preferably in less than 2 minutes. Centrifugation of the device within the holder may then be continued until full reconstitution of the dry powder has taken place. The time required will depend upon the type of powder used and the target concentration to be achieved but can be determined by simple experimentation.

It is recognised that the movement of the partition within the device on application of the centrifugal force should be between an initial stable starting position which prevents the liquid from transferring during manufacture, shipping and storage and a second defined position where the liquid transfer can take place. Once the liquid transfer from the Liquid Chamber to the Powder Chamber has taken place and the dry powder has been reconstituted the plug or plunger may be further used to expel the solution from the other end of the Powder Chamber. FIG. 2 and FIG. 5 illustrates how such a device may operate.

According to a yet further aspect the present invention provides a kit comprising:
(a) an assembly as defined hereinbefore comprising a device and holder, wherein the chambers of the device are pre-loaded with dry product and suitable diluent therefor;
(b) a dummy assembly of equivalent weight to the pre-loaded dry product containing assembly, and preferably of similar dimensions;
(c) a centrifuge wherein the rotor is adapted to receive at one end the pre-loaded assembly and at the opposite end the dummy assembly.
   Such dummy assemblies may be moulded plastic assemblies, and in a variation to the aspect hereinbefore the kit may alternatively comprise a rotor adapted to receive at one end a pre-loaded assembly as defined herein, and at the other end the rotor has an integral weight equivalent to that of the pre-loaded assembly to be inserted therein.
   As will be appreciated the kit comprising the centrifuge may additionally comprise one or more suitably adapted rotors suitable for use with different pre-loaded weights of assembly.
   The assemblies can be provided for single-use (disposable), or multiple (re-suseable) formats. For many applications a single-use, sealed assembly within sterile packaging will be preferred to maintain the integrity of the kit pre-use and the centrifuge apparatus post-use to enable efficient use of the present assemblies. Different dry products could be bar-coded or color coded with corresponding dummy assemblies or adapted rotors having integral weights therefore.
   The invention will now be described, by way of illustration only, with reference to the following examples and figures accompanying the specification.

### DESCRIPTION OF FIGURES

FIGS. 1 to 5 illustrate various aspects and features of devices, and devices within holders for use in the assemblies in accordance with the present invention and are not intended to be limiting thereupon.

For the avoidance of doubt, in FIGS. 3 to 5 the various assemblies are illustrated in cross-sectional view and show the internal arrangement thereof, and on application of centrifugal force to such assemblies when placed their pointed-ends are into the rotor of a centrifuge they will lie so that the pointed end is furthest from the centre of the rotor.

FIG. 1A illustrates a device for use in an assembly according to the invention with Liquid Chamber (1), Active Partition in the form or a seal (2) and Powder Chamber (3).

FIG. 1B illustrates how upon application of centrifugal force, the device of FIG. 1A, when placed and securely held by the holder of the rpesent assembly (also not shown) within a centrifuge (5) (not shown) hydrostatic pressure leads to rupture of the seal of the Active Partition (6) and liquid can then be transferred into the Powder Chamber (7) to start the reconstitution process.

FIG. 1C illustrates the device of FIG. 1A on removal from the centrifuge with the liquid transferred out of Liquid Chamber (8) to provide a fully reconstituted powder (9) in the Powder Chamber.

FIG. 2D illustrates a device for use in an assembly according to the invention in which the diluent in the Liquid Chamber (10) is prevented from transferring into the Powder Chamber (12) when the Active Partition (11) is in the closed position. The Active Partition (11) may be mounted on a plunger (13).

FIG. 2E illustrates application of a centrifugal force in a centrifuge (14) (not shown) to a device of FIG. 2D, whereby the Active Partition is forced downwards into a new position by the hydrostatic pressure to open up channels (16) through which the liquid (diluent) can pass from the liquid-containing to the powder-containing chamber to start the reconstitution process. The plunger (15) may be designed with a stopper (not shown)to ensure the Active Partition is prevented from moving past the most suitable position for allowing the transfer of liquid (diluent) into the Powder Chamber (17).

FIG. 2F illustrates the device of FIG. 2D on removal from the centrifuge. The Liquid Chamber (18) is empty and the Powder chamber contains a fully reconstituted powder (19).

FIG. 3 shows the use of a holder for use in the assembly according to the present invention in order to align a vial and syringe within the rotor of a centrifuge.

FIG. 3G illustrates the use of a holder for use in an assembly according to the invention with an external shape similar to that of a Falcon™ tube with the conical bottom of the holder designed to fit snuggly into the rotor of a centrifuge. As illustrated in the right hand image of 3G, the holder can be split longitudinally to allow introduction and removal of devices. The interior of the holder is designed to hold devices securely during shipping and handling. In

FIG. 3G (right hand image) it is shown how interior flanges or inserts can be used to hold a syringe securely and prevent the plunger from moving both during shipping and on application of a centrifugal force.

FIG. 3H shows how a vial and syringe are aligned within a holder (20) prior to application of a centrifugal force. In this device the liquid chamber is a vial (21) filled with liquid (22) and this is sealed with a septum (23) which forms the Active partition along with the needle (24). A compressible spacer (25) prevents the needle from coming into contact with the septum during normal handling. The syringe barrel (26) contains dry powder (27) for reconstitution. The tip of the plunger (28) provides a seal at the other end of the powder chamber and held in place by the plunger rod (29) fixed in place in the holder.

FIG. 3I shows the effect of applying a suitable relative centrifugal force to the device within the holder. The centrifugal force acting on the vial and liquid (30) leads to piercing of seal (31) by the needle as the vial is able to move towards the cone end of the holder as the spacer is compressed (32). Once the needle pierces the septum the Active Partition is activated and liquid transfers from the vial into the dry powder in the syringe (33). Typically following activation of the Active Partition transfer of the liquid into the dry powder chamber will take 1 to 2 minutes. However, the time to achieve full reconstitution in the centrifuge will depend on the type of dry powder and the target concentration of the solution.

FIG. 3J shows the device within the holder on removal from the centrifuge. The empty vial (34) is pushed back to its original position by the compressible spacer (35) and the reconstituted solution (36) is in the syringe and ready for injection.

FIG. 4 shows a dual chamber syringe within a holder to form an assembly in accordance with an embodiment of the invention which is suitable for insertion into the rotor of a centrifuge.

FIG. 4K illustrates the use of a holder (37) to secure a dual chamber syringe so that it can be placed within the rotor of a centrifuge. The part of the syringe barrel closest to the needle (38) contains the dry powder (39) and the Active Partition (40) which is a stopper valve separates it from the liquid diluent (41). A plunger stopper (43) seals the other end of the syringe and is prevented from being displaced during handling or centrifugation by flanges or inserts within the holder (43).

FIG. 4L shows the effect of applying a suitable relative centrifugal force to the dual chamber syringe within the holder. Liquid diluent is transferred upwards into the dry powder (44) as the centrifugal force acting on the stopper valve (Active Partition) generates sufficiently high hydrostatic pressure within the liquid chamber to open the channels within the stopper valve (45) and thus activate it. Liquid diluent is then displaced upwards into the dry powder chamber as the stopper valve moves downwards towards the plunger stopper. Typically following activation of the Active Partition transfer of the liquid into the dry powder chamber will take 1 to 2 minutes. However, the time to achieve full reconstitution in the centrifuge will depend on the type of dry powder and the target concentration of the solution.

FIG. 4M shows the syringe within the holder on removal from the centrifuge. The dry powder is fully reconstituted and the solution is in a syringe and ready for injection.

FIG. 4N shows the holder can be sealed within a flexible pouch that provides a sterile barrier. Sealed within the pouch the holder can still be placed within the rotor of a centrifuge and so it is possible to carry out a sterile reconstitution process to produce a ready-to-use filled syringe that can be kept for many weeks or months.

FIG. 5 shows a dual chamber cartridge within a holder suitable to provide an assembly in accordance with an embodiment of the invention which is suitable for insertion into the rotor of a centrifuge

FIG. 5O illustrates the use of a holder to secure a dual chamber cartridge containing a bypass valve to allow it to be used within the rotor of a centrifuge. The liquid chamber is located at the top of the cartridge and is sealed at the outer end with a plunger stopper (48). The lower interior end of the liquid chamber (49) is sealed with a bypass stopper (50) located above the bypass valve section (Active Partition) within the barrel of the cartridge (51). The dry powder (52) is placed in the lower chamber and the cartridge is sealed with a suitable cap (53).

FIG. 5P shows the effect of applying a suitable relative centrifugal force to the dual chamber cartridge within the holder. The plunger stopper (54) and bypass stopper are forced to move towards the bottom of the cartridge until the bypass stopper reaches the bypass valve section (55). This opens the Active partition and allows liquid to pass around the stopper and transfer into the dry powder chamber. Typically following activation of the Active Partition transfer of the liquid into the dry powder chamber will take 1 to 2 minutes. However, the time to achieve full reconstitution in the centrifuge will depend on the type of dry powder and the target concentration of the solution.

FIG. 5Q shows the cartridge within the holder on removal from the centrifuge. The dry powder is fully reconstituted and the solution is in a cartridge and ready for injection.

FIG. 5R illustrates how a cartridge could be filled with powder and diluent, placed in a holder and enclosed in packaging that provides a sterile barrier whilst in a sterile clean room. It can then be reconstituted by rotating it in a centrifuge to produce a sterile filled cartridge which is ready for injection but which may be kept for weeks or months.

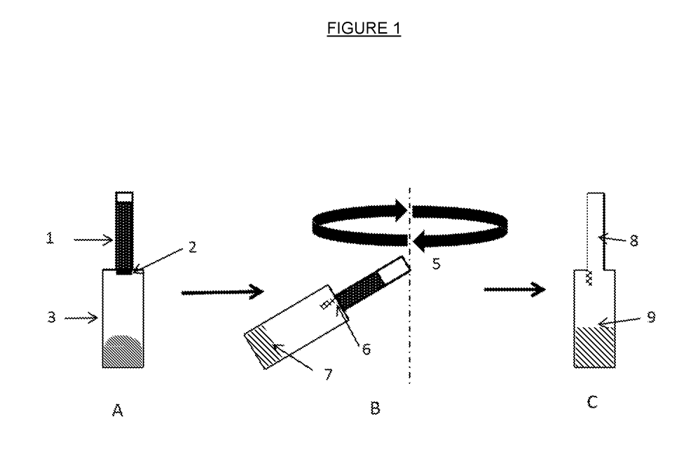

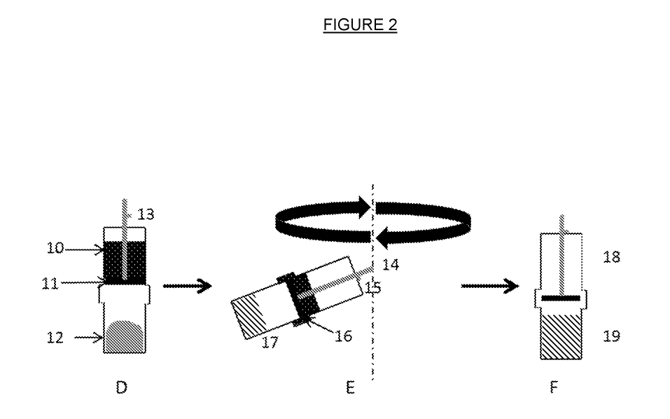

The following examples illustrate methods for the reconstitution of dry powders within assemblies in accordance with the present invention which utilise different active partitions. Such assemblies may be further modified to include one or more additional features as detailed hereinbefore and such modified assemblies are considered to be within the scope of the present invention. As such the present examples are considered to be non-limiting upon the present invention.

### EXAMPLES

In each of the following examples an ALC PK130R centrifuge (T535 4-fold swing-out rotor with P510 cups and 4 piece Falcon tube adaptor) was used to apply the centrifugal force to the assembly.

### Example 1

### Reconstitution of a Dry Powder in a Centrifuge Using an Assembly Comprising a Device Held within a Holder with an Active Partition Based on Piercing a Seal

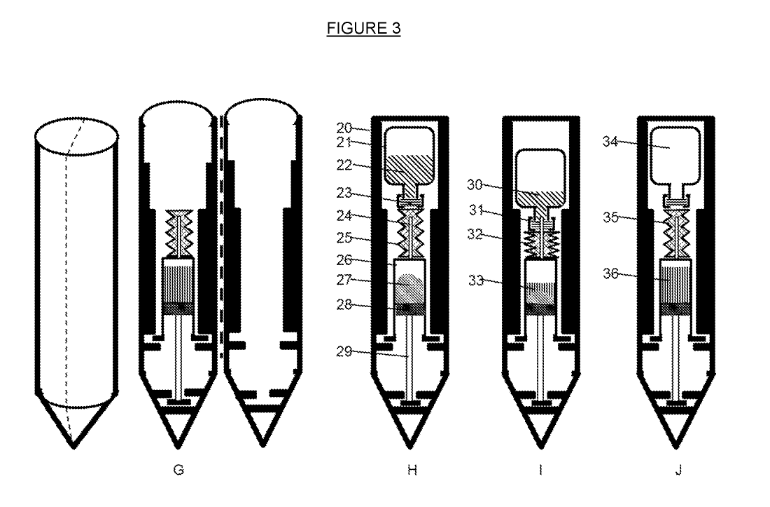

In this example, application of centrifugal force is used to trigger the piercing of a seal (Active partition) between a liquid chamber (vial) and a dry powder chamber (syringe with needle) and further to promote transfer of liquid into the dry powder chamber resulting in reconstitution to form a clear solution. The vial and the syringe with needle are secured within a holder that keeps them axially aligned whilst in the centrifuge as illustrated in FIG. 3.

A compressible spacer such as a spring or rubber-like sleeve is used to prevent the syringe needle coming into contact with the septum seal on the vial during normal handling or shipping. The assembly is placed into the rotor so that the vial (liquid chamber) is placed above the syringe (dry powder chamber) within the holder. On application of a centrifugal force the increased weight of the vial compresses the spacer and the syringe needle then pierces the septum on the vial. Liquid is then rapidly transferred into the syringe through the needle and any air displaced is simultaneously transferred into the vial. Reconstitution of the dry powder to form a clear solution then takes place.

A holder suitable for axially aligning the syringe and vial was produced as follows: a plastic Falcon tube was cut in half longitudinally to provide an outer shell with a shape suitable for insertion into the rotor of a syringe; a polystyrene insert was shaped to provide coaxial cylindrical volumes one above each other into which the vial and syringe (including needle) could be placed. A further cylindrical insert was produced to prevent the plunger from moving within the syringe whilst it was in the holder. A BD Hypak pre-fillable syringe with a standard needle guard comprising an outer plastic cylinder and an inner rubber-like polymer insert (BD™ Rigid Needle Shield (RNS) was selected. The needle guard was removed and modified. The outer plastic cylinder of the needle guard was cut to expose the insert and shorten it by 40% and the inner rubber-like insert of the needle guard was cut to shorten it by 30%. The so-modified needle guard was then placed back onto the needle. Dry powder (lyophilised or precipitated protein), at a level of from 1-50 mg was filled into the syringe, the plunger was inserted and the syringe was located into the holder.

A sealed vial containing 1-2 ml of the diluent was placed into the holder with its sealed end lying adjacent to the modified needle guard. The outer shells of the holder were secured together and the assembly was placed into the rotor of a centrifuge so that the vial was placed above the syringe.

On application of a suitable relative centrifugal force (RCF greater than about 2500 xg and less than 4000 xg) the vial compressed the rubber-like section of the modified needle guard sufficiently to expose the needle and pierce the vial seal. The liquid diluent was then transferred into the syringe leading to reconstitution of the dry powder. Advantageously the reconstitution is carried out without formation of foam within the syringe so that all of the solution is available for injection.

### Example 2

### Reconstitution of a Dry Powder in a Centrifuge Using a Device Held within a Holder with an Active Partition Based on a Stopper Valve

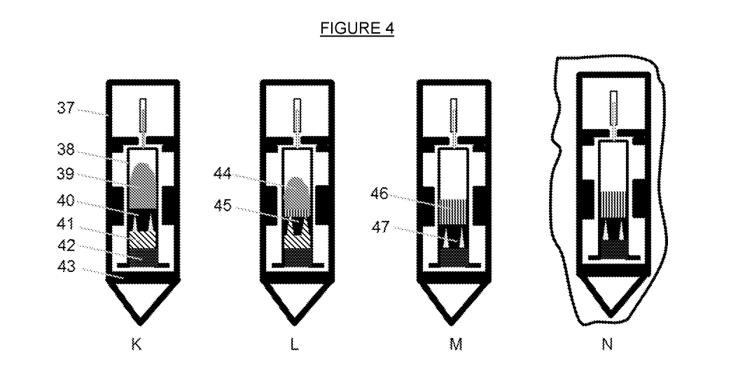

In this example, application of centrifugal force is used to generate sufficient hydrostaticpressure, on one side of a stopper valve (Active Partition), to open pre-existing channels within the stopper and allow the liquid to pass from the liquid chamber to the dry powder chamber. Surprisingly the optimal configuration for this assembly was found to be with the device placed in the holder so that the dry powder chamber is placed above the liquid chamber, within the centrifuge, as illustrated in FIG. 4.

The stopper valve (Active Partition) sits between the powder and the liquid. Under normal conditions the channels, within the stopper valve, remain firmly closed so the two components are isolated from each other. When a suitable centrifugal force is applied, the stopper valve presses down onto the liquid and the hydrostatic pressure generated forces open the channels within the valve. The liquid is then transferred upwards through the channels and into the dry powder chamber whilst the stopper valve moves downwards thereby displacing the liquid and it continues until it contacts with the fixed plunger or stopper at the opposite end of the liquid chamber.

Test stopper valves were manufactured by cutting two narrow channels into standard commercially available syringe plungers using a scalpel. In the test the plungers were removed from BD Hypak Syringes, (2-10 ml). Lyophilised or precipitated protein powder (˜10 mg) was placed into the needle end of the barrel of a BD Hypak SCF Syringe and a modified plunger/test stopper valve was inserted into the barrel using a needle to expel the air (as known in the art). Liquid diluent (˜1 ml) was introduced and the end of the syringe stoppered with a stopper made from a conventional BD plunger. The BD Hypak Syringe containing the test stopper valve was placed in a clear plastic cylindrical holder and the assembly was inserted into a bench-top centrifuge with the syringe needle pointing upwards. On application of a suitable relative centrifugal force (2000 xg) the diluent was transferred into the dry powder and reconstitution took place producing a clear protein solution.

Enclosure of the described assembly in a sterile packaging such as a Tyvek pouch is straightforward and does not affect the functionality of the Active Partition based on a stopper valve within a centrifuge. The assembly described can therefore be used to carry out sterile reconstitution of dry powders with minimal manual intervention. Once the dry powder has been reconstituted the assembly is equivalent to a ready to inject prefilled syringe held in a sterile packaging.

Advantageously an Active Partition based on a stopper valve as demonstrated here can be manufactured from standard materials used to form plungers and stoppers in clinically approved syringes and cartridges. As demonstrated the stopper valves can also be used to convert standard syringes or cartridges into a dual chamber system. This leads to reduced costs and means all the device components in contact with the dry powder and resultant solution will have already been clinically approved.

### Example 3

### Reconstitution of a Dry Powder in a Centrifuge Using a Device Held within a Holder with an Active Partition Based on a Bypass Valve

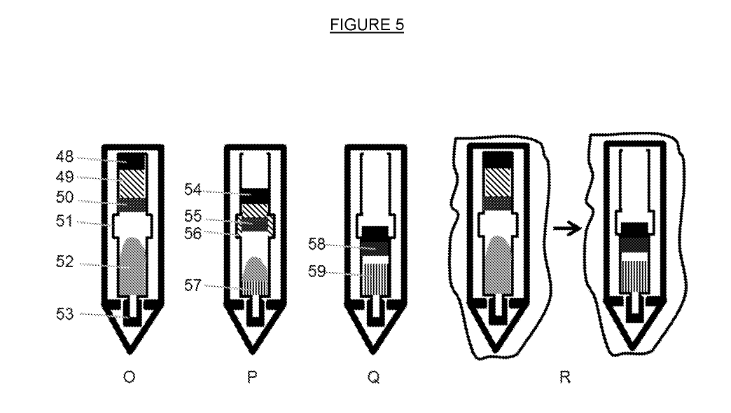

In this example, application of centrifugal force is used to move an internal stopper (Active Partition) from a position where it prevents liquid diluent transferring into the dry powder into an alternative position where liquid is able to bypass the stopper (bypass valve) and transfer into the dry powder as illustrated in FIG. 5.
Advantageously the use of centrifugal force to trigger movement of the stopper into the bypass valve enables the device to be significantly smaller and cheaper to manufacture than manually operated designs currently available.
A Vetter dual chamber bypass valve syringe (Vetter LyoJect® 5.0 mL diluent for Reconstitution) was modified by removing the external tabs and the screw plunger to make it smaller and enable it to be introduced into a cylindrical holder and placed into the rotor of a bench top centrifuge. Lyophilised or precipitated protein dry powder, 4-40 mg, was placed in the lower chamber of the syringe and aqueous diluent, about 4 mg, was placed in the upper chamber. A range of centrifugal forces were applied to the filled syringe within the centrifuge. It was found that if sufficient centrifugal force (i.e. greater than about 1000 xg) was applied to the syringe the internal stopper (Active Partition) lying between the liquid and dry powder moved downwards into the bypass valve section of the syringe barrel allowing the liquid to flow into the chamber containing the dry powder as illustrated in FIG. 5.
If the applied centrifugal force was too great (i.e. more than about 2000 xg) the stopper was found to move very quickly through the bypass valve section and then reform a seal with the barrel above the dry powder such that minimal transfer of liquid into the powder occurred. Thus, surprisingly it was discovered that there is an optimal range for the centrifugal force (1000-2000 xg) that needs to be applied to the syringe, within the holder, to ensure rapid transfer of the liquid into the powder (<2 min) and to start the reconstitution process. Using the Vetter LyoJect® syringe the optimal relative centrifugal force was found to be around 1250 xg which is easily accessible using a bench-top centrifuge. For other devices containing a bypass valve such as a the Vetter VL-K dual chamber cartridge the optimal centrifugal force required to open the valve will be different but suitable relative centrifugal forces will preferably lie in the range 400-4000 xg.
The holder containing a filled dual chamber syringe was placed within a heat-sealed Tyvek pouch which provides a sterile barrier and introduced into the rotor of a centrifuge. On application of a relative centrifugal force of 1250 xg the dry powder was fully reconstituted within the syringe whilst it was packaged inside the Tyvek pouch.
These experiments clearly demonstrate that centrifugal force can be utilised to activate the bypass valve within a dual chamber device that has been placed within an appropriate holder and this can be used for the reconstitution of a dry powder with a liquid diluent. The process can also be carried out when the device within the holder is further sealed within a Tyvek pouch. It is known in the art that such a Tyvek pouch provides a sterile barrier for all components within the pouch. Hence, if the device is filled, placed in the holder and sealed in the barrier packaging in a sterile environment using methods known in the art then the reconstitution of a powder to form a sterile solution can be carried out using the described device within a holder and with minimal manual intervention. The centrifugal force required (>1000 xg) is much greater than would be experienced by devices such as syringes or cartridges during normal shipping and so the reconstitution would only be initiated when required.

1-15. (canceled)

16. Assembly for reconstitution of a dry product in a centrifuge comprising a holder for a device and a device wherein the holder is adapted to fit within a centrifuge, and wherein the device comprises a chamber containing a liquid and a further chamber containing a dry powder wherein said chambers are separated by an active partition and wherein the active partition is activated upon application of a suitable centrifugal force and liquid is transferred into the chamber containing the dry powder.

17. An assembly according to claim 16 wherein said chambers of the device are detachable from one another or are formed as a single structure.

18. An assembly according to claim 16 wherein said active partition comprises a seal that is ruptured or pierced upon activation by a centrifugal force.

19. The assembly according to claim 16 wherein channels suitable for transfer of said liquid are opened in said active partition upon activation by a centrifugal force.

20. An assembly according to claim 16 wherein the position of said active partition is changed upon activation by a centrifugal force.

21. An assembly according to claim 16 wherein said active partition operates as a valve upon activation by a centrifugal force.

22. An assembly according to claim 16 wherein said holder is adapted to enclose said device and wherein the holder is suitable for use in the rotor of a centrifuge and wherein the holder is adapted to prevent activation of the activation partition until the assembly is placed in the rotor of a centrifuge and a suitable centrifugal force applied.

23. An assembly according to claim 16 wherein on application of a suitable centrifugal force said holder enables components of the device to move relative to each other to activate said active partition but holds the remaining components in a constant relative position.

24. An assembly according to claim 16 wherein said active partition can be activated with a relative centrifugal force in the range of from 50 xg to 5000 xg.

25. An assembly according to claim 16 wherein said active partition can be activated with a relative centrifugal force in the range of from 400 xg to 4000 xg.

26. An assembly according to claim 16 wherein the device comprises a syringe or a cartridge or a vial or an injection device.

27. An assembly according to claim 16 wherein the device and holder are enclosed within sterile packaging.

28. An assembly according to claim 16 wherein reconstitution of a dry product to form a sterile solution is activated by application of a centrifugal force.

29. A method for the reconstitution of a dry product with a suitable diluent in a reconstitution device comprising:
(a) introduction of a suitable diluent for the dry product to be reconstituted into a chamber of said device suitable for containing liquid;
(b) introduction of dry product to be reconstituted into a further chamber of said device suitable for containing dry product;
(c) placing the device with diluent and dry product containing chambers into a suitable holder and optionally placing said device-containing holder within suitable packaging for the provision of a sterile barrier;
(d) placing the holder containing the device in the rotor of a centrifuge and rotating the centrifuge; and
wherein said chambers are separated by an active partition and wherein upon application of a suitable centrifugal force the active partition is activated and diluent is transferred into the dry product containing chamber.

30. The method of claim 29 wherein the device within the holder is an assembly according to claim 16.

31. Use of the method of claim 29 for the reconstitution of dry pharmaceutical or biopharmaceutical formulations, or sterile reconstitution of dry pharmaceutical or biopharmaceutical formulations, wherein said formulations include lyophilised powders, spray-dried powders and precipitated powders.
